(19) United States
(12) Patent Application Publication
(10) Pub. No.: US 2018/0125674 A1
(43) Pub. Date: May 10, 2018

(54) VERTEBRAL BODY EXPANDING DEVICE AND TOOL SET FOR OPERATING THE SAME

(71) Applicants: Hsi-Yi Liu, Taoyuan (TW); Shih-Hsuan Shen, Kaohsiung (TW)

(72) Inventors: Hsi-Yi Liu, Taoyuan (TW); Shih-Hsuan Shen, Kaohsiung (TW)

(21) Appl. No.: 15/643,446

(22) Filed: Jul. 6, 2017

(30) Foreign Application Priority Data
Nov. 9, 2016 (TW) 105136522

(51) Int. Cl. A61F 2/44; A61F 2/46

(52) U.S. Cl. CPC A61F 2/4425; A61F 2002/30235; A61F 2/4611

(57) ABSTRACT

A vertebral body expanding device includes a bolt, a first nut, a second nut, a first supporting member, a second supporting member, a third supporting member, a fourth supporting member, a first expanding member and a second expanding member. The first nut and the second nut engage the bolt by two opposite screw threads. The first and third supporting members are pivoted to upper and lower ends of the first nut respectively, and the second and fourth supporting members are pivoted to upper end and lower ends of the second nut respectively. The first and second supporting members are pivoted to a lateral surface of the first expanding member, and the third and fourth supporting members are pivoted to a lateral surface of the second expanding member. The first and second nuts move toward or away from each other to retract or expand the first and second expanding members.

### FIELD OF THE INVENTION

The present invention relates to a vertebral body expanding device and a tool set for operating the vertebral body expanding device, and more particularly to a vertebral body expanding device for expanding vertebral bodies and a tool set for operating the vertebral body expanding device.

### BACKGROUND OF THE INVENTION

More and more patients are suffering from compressional vertebral fracture caused by osteoporosis. Although surgical techniques and tools for compressional vertebral fracture have gradually improved, surgical tools for such fracture still have many clinical problems, such as excessive damage to bone material of the vertebral body, re-collapse of vertebral height, insufficient height and angle rebuild for vertebral collapse, and leakage of bone cement etc., leading to failure of the surgeries.

Existing surgical operations for compressional vertebral fracture include vertebroplasty, kyphoplasty and vertebral expandable implant. Vertebral expandable implant is a newer surgical approach, which uses a metal expander to expand the vertebral body along a longitudinal axial direction of the spine and fills the expanded spine with bone cement to fix and thus restore the collapsed vertebral body.

In spite of the described clinical treatments for compressional vertebral fracture, many problems still exist after the surgerical operations. For example, vertebroplasty cannot restore vertebral height and may cause leakage of bone cement. Kyphoplasty uses a balloon to support the collapsed vertebral body; however, the balloon is easily deflated during inflation, and the vertebral body may re-collapse when the balloon is removed. Vertebral expandable implant uses a metal device to support the collapsed vertebral body; however, the supporting effect is inconsistent; for example, the vertebral expandable implant often causes insufficient restored vertebral height and thus insufficient space for bone cement, or even causes imprecise restored vertebral height which may result in deformity of spinal curvature.

### SUMMARY OF THE INVENTION

An objective of the invention is to provide a vertebral body expanding device and a tool set for operating the vertebral body expanding device. The vertebral body expanding device of the present invention can expand the vertebral body safely and firmly and thus create a space for filling bone cement safely. In addition, the vertebral body expanding device of the present invention can prevent leakage of the bone cement effectively, and medical institutions may utilize the vertebral body expanding device of the present invention to adjust the required restored height of the vertebral body according to the level of spinal collapse and adjust the angle of spinal according to the curvature of spinal deformity. The vertebral body expanding device of the present invention has a shorter length when retracted. The vertebral body expanding device can continuously adjust expansion height and provides stronger supporting strength.

An embodiment of the vertebral body expanding device of the present invention includes a bolt, a first nut, a second nut, a first supporting member, a second supporting member, a third supporting member, a fourth supporting member, a first expanding member and a second expanding member. The bolt includes outer clockwise screw threads and outer counter-clockwise screw threads adjacent to the clockwise screw threads. The first nut includes inner clockwise screw threads and is joined to the bolt by the inner clockwise screw threads engaging the outer clockwise screw threads. The second nut includes inner counter-clockwise screw threads and is joined to the bolt by the inner counter-clockwise screw threads engaging the outer counter-clockwise screw threads. The first supporting member includes one end movably connected to the first nut. The second supporting member includes one end movably connected to the second nut. The third supporting member includes one end movably connected to the first nut. The fourth supporting member includes one end movably connected to the second nut. The first expanding member includes a lateral surface movably connected to another end of the first supporting member and another end of the second supporting member. The second expanding member includes a lateral surface movably connected to another end of the third supporting member and another end of the fourth supporting member. The bolt is rotated to allow the first nut and the second nut to move in opposite directions along the bolt so that movements of the first supporting member, the second supporting member, the third supporting member and the fourth supporting member drive the first expanding member and the second expanding member to move in opposite directions relative to the lateral surfaces.

In another embodiment, the end of the first supporting member is pivoted to an upper end of the first nut, the end of the second supporting member is pivoted to an upper end of the second nut, the end of the third supporting member is pivoted to a lower end of the first nut, and the end of the fourth supporting member is pivoted to a lower end of the second nut.

In another embodiment, the vertebral body expanding device further includes a transmission member fixed to one end of the bolt. The transmission member includes an interfering structure for an external tool to engage therewith to exert force on the transmission member so as to rotate the bolt about an axis.

In another embodiment, the interfering structure is disposed on an outer surface of the transmission member.

In another embodiment, the interfering structure is disposed on a lateral wall of a recessed hole formed on the transmission member.

In another embodiment, the vertebral body expanding device further includes a guiding member fixed to the other end of the bolt. The guiding member has a conical shape different from a shape of the other end of the bolt.

In another embodiment, the first expanding member includes two lateral sides extending toward the second expanding member, and the second expanding member includes two lateral sides extending toward the first expanding member so as to form an enclosed space.

In another embodiment, the bolt is hollow and further includes at least one guiding groove.

In another embodiment, the vertebral body expanding device is made of a material selected from the group consisting of polyether ether ketone (PEEK) and titanium alloy.

In another embodiment, the first expanding member comprises at least one rib disposed on an outer surface thereof, and the second expanding member comprises at least one rib disposed on an outer surface thereof.

The present invention also provides a tool set for operating the vertebral body expanding device of the present invention. An embodiment of the tool set of the present invention includes a rotational member having one end connected to the vertebral body expanding device, a sleeve surrounding the rotational member and having one end joined to the rotational member and another end connected to the vertebral body expanding device, and a grip surrounding the rotational member and the sleeve. The rotational member is rotated to allow the sleeve to rotate so as to rotate the bolt of the vertebral body expanding device. The grip does not rotate with the rotational member and the sleeve.

The vertebral body expanding device and tool set of the present invention supports vertebral bodies safely and firmly and thus forms a space for filling bone cement safely. The two half-enclosed expanding members can prevent leakage of bone cement effectively. By utilizing stable movements of the first nut and the second nut along the bolt, medical institutions may adjust the desired restored height according to the level of vertebral collapse and adjust the angle of the spine according to the curvature of spinal deformity. Rotation of two opposite screw threads enables the vertebral body expanding device of the present invention to be shorter in length when retracted, to adjust the expansion height continuously, and to have stronger supporting strength.

### BRIEF DESCRIPTION OF THE DRAWINGS

The present invention will become more readily apparent to those ordinarily skilled in the art after reviewing the following detailed description and accompanying drawings, in which:

FIG. 1A is a schematic view of a vertebral body expanding device of the present invention when retracted;

FIG. 1B is a schematic view of a vertebral body expanding device of the present invention when expanded;

FIG. 2 depicts a vertebral body expanding device of the present invention when placed in a vertebral body;

FIG. 3 depicts a vertebral body expanding device of the present invention expanded in a vertebral body;

FIG. 4 is an exploded schematic view of an embodiment of a tool set of the present invention;

FIG. 5 depicts a tool set of the present invention operating a vertebral body expanding device of the present invention;

FIG. 6 depicts an outer surface of an expanding member of a vertebral body expanding device of the present invention; and

FIG. 7 is a schematic view of bidirectional screw threads and guiding grooves of a bolt of a vertebral body expanding device of the present invention.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

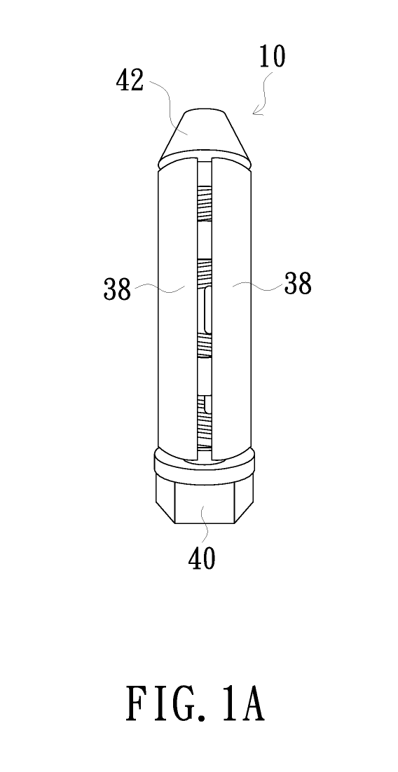

Referring to FIGS. 1A and 1B, the present invention relates to a vertebral body expanding device 10. The vertebral body expanding device 10 includes a bolt 30, a first nut 32, a second nut 34, a first supporting member 3602, a second supporting member 3604, a third supporting member 3606, a fourth supporting member 3608 and two expanding members 38 (including a first expanding member and a second expending member).

The bolt 30 includes an outer clockwise screw thread 3002 and an outer counter-clockwise screw thread 3004 adjacent to the clockwise screw thread 3002. The vertebral body expanding device 10 further includes a transmission member 40 fixed to an end of the bolt 30. The transmission member 40 has an interfering structure for an external tool to engage therewith to exert force on the transmission member 40 to rotate the transmission member 40 so as to rotate the bolt 30 about a longitudinal axis.

The vertebral body expanding device 10 further includes a guiding member 42 fixed to another end of the bolt 30. The guiding member 42 has a conical shape different from the shape of the other end of the bolt 30. The guiding member 42 facilitates insertion of the vertebral body expanding device 10 into a collapsed vertebral body. The bolt 30 includes at least one guiding groove 3006. In this embodiment, the bolt 30 includes three guiding grooves 3006, which facilitate flow of the bone cement into the vertebral body from the bolt 30 and binding of the bone cement with the vertebral body expanding device 10. The vertebral body expanding device 10 bond with the bone cement has a stronger structure.

The first nut 32 includes an inner clockwise screw thread, and the second nut 34 includes an inner counter-clockwise screw thread. The first nut 32 and the second nut 34 are joined to the bolt 30 by engagement of the inner clockwise screw thread of the first nut 32 with the outer clockwise screw threads 3002 of the bolt 30 and by engagement of the inner counter-clockwise screw thread of the second nut 34 with the outer counter-clockwise screw thread 3004 of the bolt 30. Therefore, when the bolt 30 is rotated, the first nut 32 and the second nut 34 move in opposite directions along the bolt 30 to move toward or away from each other.

The first supporting member 3602 includes an end movably connected to the first nut 32, the second supporting member 3604 includes an end movably connected to the second nut 34, the third supporting member 3606 includes an end movably connected to the first nut 32, and the fourth supporting member 3608 includes an end movably connected to the second nut 34. The movable connection can be a pivot connection structure or a connection structure in which elements are movable by deformation. In this embodiment, the end of the first supporting member 3602 is pivoted to the first nut 32, the end of the second supporting member 3604 is pivoted to the second nut 34, the end of the third supporting member 3606 is pivoted to the first nut 32, and the end of the fourth supporting member 3608 is pivoted to the second nut 34. Pivot points of the above-described elements are connected by a shaft mechanism. Since the related technology is known and used in the application, descriptions of the shaft mechanism or the deformation-based connection structure are thus omitted. It is further illustrated in FIGS. 1A and 1B that the end of the first supporting member 3602 is movably connected to an upper end of the first nut 32, the end of the second supporting member 3604 is movably connected to an upper end of the second nut 34, the end of the third supporting member 3606 is movably connected to a lower end of the first nut 32, and the end of the fourth supporting member 3608 is movably connected to a lower end of the second nut 34. The movable connection can be pivot connection or connection in which elements are movable by deformation.

One of the two expanding members 38 includes a lateral surface pivoted to another end of the first supporting member 3602 and another end of the second supporting member 3604. The other expanding member 38 includes a lateral surface pivoted to another end of the third supporting member 3606 and another end of the fourth supporting member 3608. Pivot points of the above-described elements are connected by a shaft mechanism. Since the related technology is known and used in the application, description of the shaft mechanism is thus omitted.

Therefore, the first supporting member 3602, the second supporting member 3604, the third supporting member 3606 and the fourth supporting member 3608 lift the expanding members 38 when the first nut 32 and the second nut 34 move away from each other; and the first supporting member 3602, the second supporting member 3604, the third supporting member 3606 and the fourth supporting member 3608 move toward the bolt 30 to pull the expanding members 38 toward the bolt 30 when the first nut 32 and the second nut 34 move toward each other.

Each of the expanding members 38 has two lateral sides extending toward the other expanding member 38 to form a semi-cylindrical structure. When the expanding members 38 are lifted, they form a space enclosing the bolt 30. Therefore, when bone cement is filled in, the expanding members 38 prevent leakage of bone cement. When the expanding members 38 retract, they form a cylindrical shape similar to a rod, which has a minimal volume to reduce damage to the vertebral body 20.

When the bolt 30 is rotated, the first nut 32 and the second nut 34 move in opposite directions so as to move the first supporting member 3602, the second supporting member 3604, the third supporting member 3606 and the fourth supporting member 3608 to drive the expanding members 38 to move in opposite directions relative to the lateral surfaces. When the first nut 32 and the second nut 34 move toward each other, the vertebral body expanding device 10 retracts. When the first nut 32 and the second nut 34 move away from each other, the first supporting member 3602, the second supporting member 3604, the third supporting member 3606 and the fourth supporting member 3608 lift the expanding members 38 to expand the collapsed vertebral body, and the bone cement can be filled into the space expanded by the expanding members 38 in the vertebral body.

The vertebral body expanding device 10 can be made of polyether ether ketone (PEEK) or titanium alloy. Because PEEK has a material property similar to that of bones, the expanding members 38 of the vertebral body expanding device 10 are preferably made of PEEK to prevent bone loss due to stress shielding effect. In addition, the first supporting member 3602, the second supporting member 3604, the third supporting member 3606 and the fourth supporting member 3608 can be made of titanium alloy to enhance structural strength, so that the vertebral body expanding device 10 can expand the collapsed vertebral body 20 firmly.

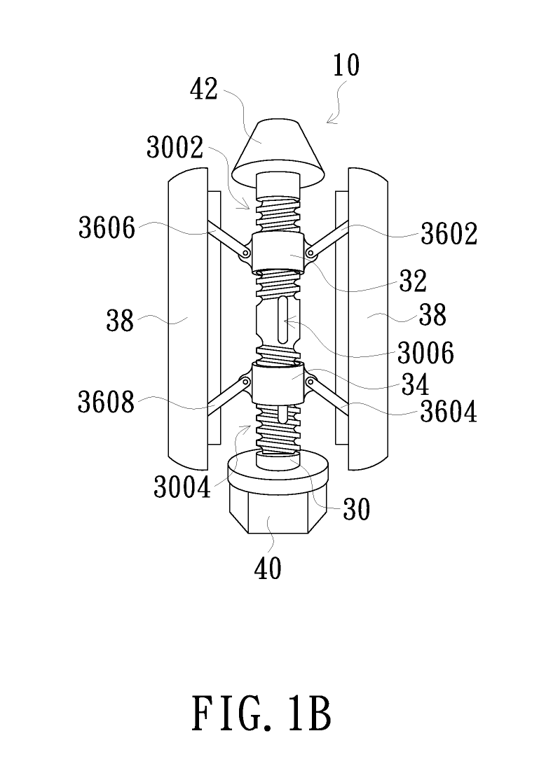

Referring to FIG. 2, the vertebral body 20 of FIG. 2 is a collapsed vertebral body. The vertebral body expanding device 10 is placed into the vertebral body 20 and is ready to be expanded by an external tool for operating the transmission member 40. The retracted vertebral body expanding device 10 is small and has a bullet-like shape. The vertebral body expanding device 10 further includes a conical guiding member 42. The guiding member 42 guides the vertebral body expanding device 10 to enter the vertebral body and reduce damage to the vertebral body.

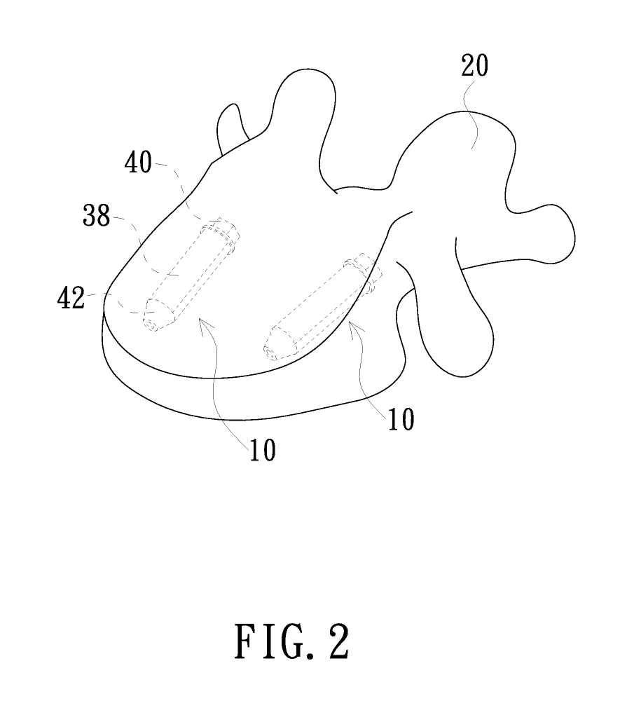

Referring to FIG. 3, in which two vertebral body expanding devices 10 are placed into the vertebral body 20. The amount of the vertebral body expanding devices 10 placed into the vertebral body 20 may vary depending on actual requirements.

When the vertebral body expanding devices 10 have been placed into the vertebral body 20, a tool is inserted into the vertebral body 20 to expand the vertebral body expanding devices 10. The tool rotates the bolt 30 to move the first nut 32 and the second nut 34 away from each other. The first supporting member 3602, the second supporting member 3604, the third supporting member 3606 and the fourth supporting member 3608 lift the expanding members 38 so as to expand the vertebral body 20. The vertebral body 20 is restored to its original height by the vertebral body expanding devices 10, and the vertebral height can be fine tuned to a precise value according to the requirement. At this time, the expanded vertebral body 20 is full of spaces and pores for bone cement to be filled into. When the bone cement is hardened, the vertebral height of the vertebral body 20 is maintained at a desired normal value.

Since the expansion of the vertebral body expanding devices 10 is controlled by two screw threads of opposite directions, expansion of the vertebral body expanding devices can be precisely tuned. In addition, if after expansion of the device but prior to filling of the bone cement, the vertebral body expanding device 10 is damaged accidentally and needs to be replaced, the vertebral body expanding device 10 can be retracted and removed from the vertebral body 20, so that another vertebral body expanding device 10 can be used to replace the damaged one.

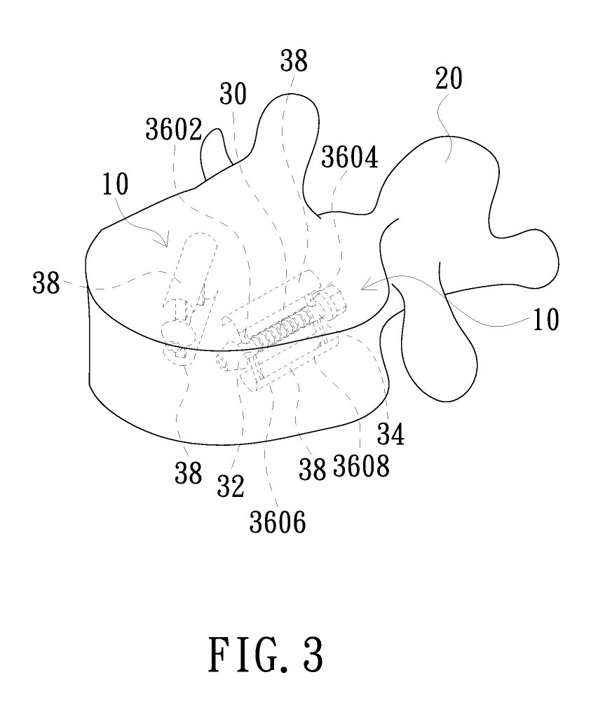

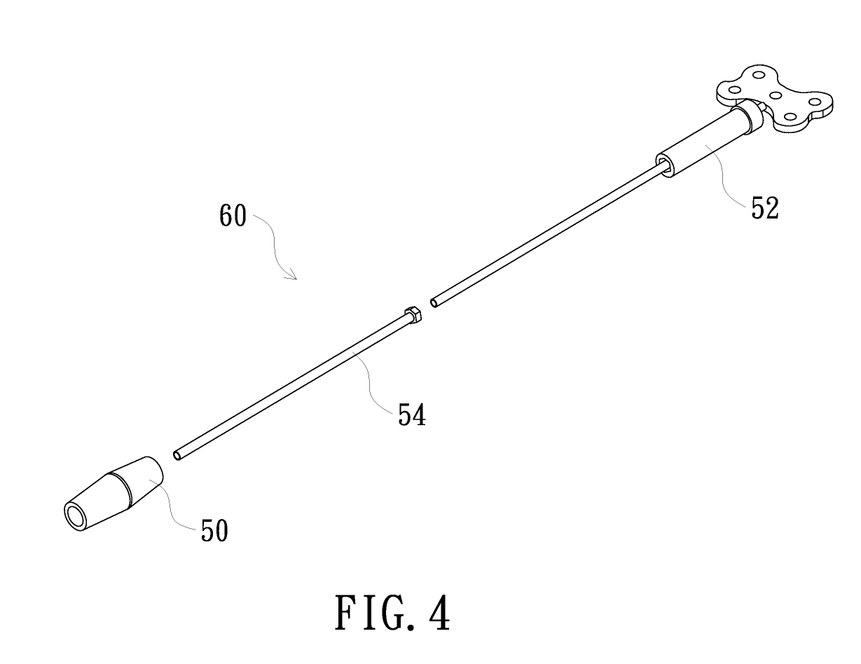

Referring to FIGS. 4 and 5, a tool set 60 is an embodiment of the external tools for expanding or retracting the vertebral body expanding device 10. The tool set 60 includes a grip 50, a sleeve 54, and a rotational member 52. The sleeve 54 is connected to the rotational member 52 and surrounds the rotational member 52. The sleeve 54 includes an upper end connected to the rotational member 52; the sleeve 54 is rotated along with the rotational member 52, but may be pulled down and detached from the rotational member 52. The rotational member 52 includes a lower end, and the sleeve 54 includes a lower end. The lower end of the rotational member 52 and the lower end of the sleeve 54 are commonly connected to the transmission member 40 of the vertebral body expanding device 10. The grip 50 is provided for a user to hold the tool set 60. The grip 50 surrounds the sleeve 54 and the rotational member 52. When the user rotates the rotational member 52, the rotational member 52 and the sleeve 54 rotate the transmission member 40 of the vertebral body expanding device 10 so as to rotate the bolt 30. However, the grip 50 does not rotate with the rotational member 52 and the sleeve 54.

When the vertebral body expanding device 10 is expanded, the rotational member 52 and the grip 50 are detached from the sleeve 54 that is still connected to the vertebral body expanding device 10. The bone cement is poured into the sleeve 54 and thus flows through the sleeve 54, the transmission member 40 and the bolt 30 to enter the vertebral body 20. The bone cement flows out of the bolt 30 through the guiding groove 3006 to fill the space of the vertebral body 20.

The lower end of the rotational member 52 of the tool set 60 is joined to the interfering structure of the transmission member 40, thereby transferring force to the bolt 30 to rotate the bolt 30. The interfering structure is disposed on an outer surface of the transmission member 40 for the lower end of the rotational member 52 to join thereto and exert force thereon. For example, if the interfering structure has a hexagonal shape, the end of the tool set 60 must include a hexagonal hole to fit the hexagonal shape of the interfering structure. In addition, the interfering structure can also be disposed on a lateral wall of a recessed hole. For example, if the interfering structure is a hexagonal recessed hole, the end of the tool set 60 must include a hexagonal shape to fit the hexagonal recessed hole of the interfering structure.

In addition, the transmission member 40 can also prevent bone cement from leaking through the rear end of the vertebral body expanding device 10. Therefore, the transmission member 40 may have a diameter similar to the diameter of the retracted vertebral body expanding device 10, so that the leakage of bone cement can be prevented effectively.

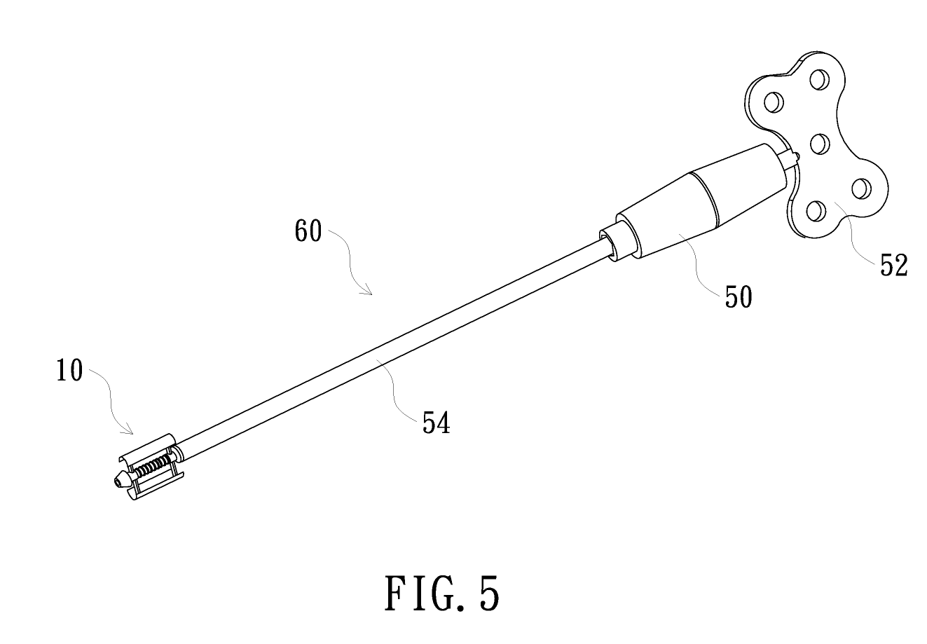

Referring to FIG. 6, several ribs 70 are disposed on an outer surface of the expanding member 38 to generate friction against the vertebral body 20 so as to enable the expanding members 38 to prop against the vertebral body 20 stably and thus prevent the vertebral body expanding device 10 from rotating in the space of the vertebral body 20. FIG. 6 shows three ribs 70 disposed on the expanding member 38.

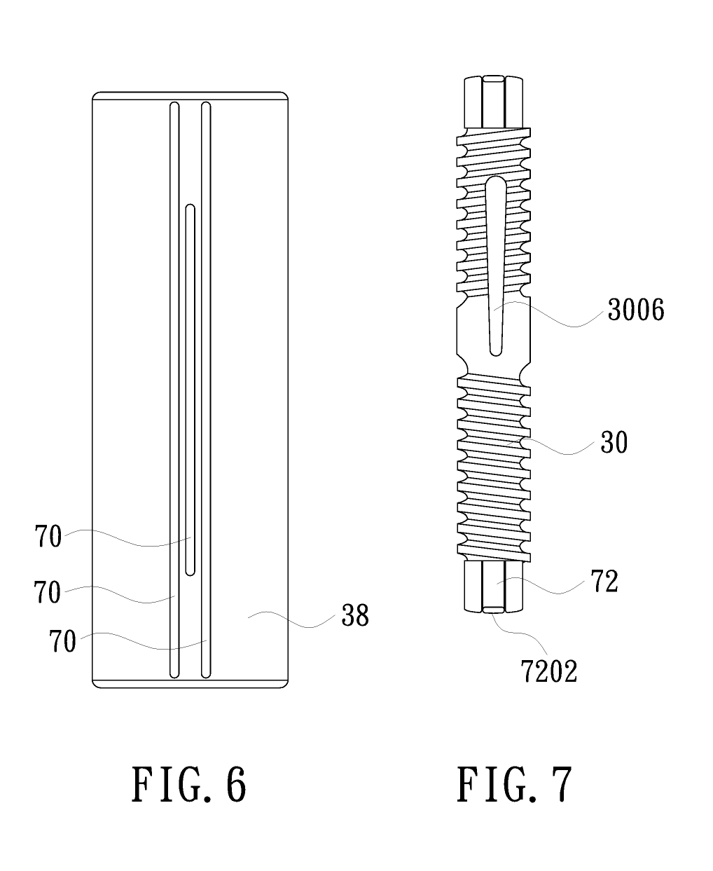

Referring to FIG. 7, the bolt 30 is hollow and has a tubular shape. The bone cement can be conducted into the tubular bolt 30. The guiding groove 3006 is designed based on the bone cement and enables the bone cement to flow into the vertebral body 20 and fill the space within the vertebral body 20 completely. When the bone cement is hardened, the structure becomes more stable. Therefore, the guiding grooves 3006 can be formed in four directions on the outer periphery of the bolt 30 and connected to an inner space of the bolt 30. Each of the guiding grooves 3006 has a larger end near a guiding end of the bolt 30 and a smaller end near the other end different from the guiding end of the bolt 30. The bone cement flows into the space of the vertebral body 20 through the larger end of the guiding grooves 3006 and fill the rear portion of the space first to form a stable and reinforced structure and thus prevent the bone cement from leaking through the front portion of the vertebral body 20 before the bone cement fills the inner space of the vertebral body 20 completely.

To facilitate production and assembly, the bolt 30, the transmission member 40 and the guiding member 42 are preferably manufactured separately and may be assembled quickly. Therefore, spring latches 72 as shown in FIG. 7 are formed at two ends of the bolt 30. The spring latches 72 are formed by parallel cutting each end of the bolt 30 twice. The spring latches 72 may be thinner to provide spring force. Each spring latch 72 has a flange 7202 at an edge thereof. The transmission member 40 and the guiding member 42 have holes fitting the spring latches 72. When the ends of the bolt 30 are inserted into the holes of the transmission member 40 and the guiding member 42, the spring latches 72 deforms to facilitate the insertion, and the flange 7202 engages the hole to firmly join the transmission member 40 and the guiding member 42 to two ends of the bolt 30 respectively. Therefore, manufacturing, processing and assembly thereof are greatly facilitated.

The embodiment of the vertebral body expanding device 10 of FIG. 1B includes a pair of the expanding members 38 corresponding to two nuts 32 and 34 and four supporting members 3602, 3604, 3606 and 3608. In another embodiment, the vertebral body expanding device includes two pairs of the expanding members (four expanding members), four nuts, and eight supporting members. When the vertebral body expanding device is expanded and viewed from an axial direction of the vertebral body expanding device, it is X-shaped. Four expanding members provide larger supporting strength. In other embodiments, the amount of the expanding members can be increased, such as to four, six, eight, and so on, depending on the collapse level of the vertebral body 20. Therefore, the vertebral body expanding device of the present invention has a structure with good expandability.

In addition, in another embodiment, when the vertebral body expanding device 10 retracts, the adjacent expanding members overlap each other at their edges. Therefore, when the expanding members are expanded, the expanding members form a large enclosed area for the bone cement, thus having excellent leakage-proof effect on the bone cement and providing stable support for the vertebral body 20. In another embodiment, the expanding members have different sizes, some of which are larger or smaller than the others. The sizes of the expanding members may vary depending on the collapse level of the vertebral body 20.

The present invention provides a vertebral body expanding device 10 and a tool set 60 for operating the vertebral body expanding device 10 to firmly and safely expand the vertebral body 20 and generate a space for bone cement filling. The two half-enclosed expanding members 38 can prevent leakage of the bone cement effectively. By utilizing stable movements of the first nut 32 and the second nut 34 along the bolt 30, medical institutions may adjust the desired restored height of the vertebral body 20 according to the level of vertebral collapse and adjust the angle of the spine according to the curvature of spinal deformity. Rotation of two opposite screw threads enables the vertebral body expanding device 10 of the present invention to have a short length when retracted to prevent excessive damage to the vertebral body, to adjust the expansion height continuously and to have stronger supporting strength. Therefore, the vertebral body expanding device 10 of the present invention can effectively improve the symptoms caused by vertebral collapse, effectively alleviate pain, restore vertebral height, prevent the vertebral body from recollapse and avoid deformity the spinal curvature.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A vertebral body expanding device, comprising:
a bolt, comprising an outer clockwise screw thread and an outer counter-clockwise screw thread adjacent to the clockwise screw thread;
a first nut, comprising an inner clockwise screw thread and joined to the bolt by engagement of the inner clockwise screw thread of the first nut with the outer clockwise screw thread of the bolt;
a second nut, comprising an inner counter-clockwise screw thread and joined to the bolt by engagement of the inner counter-clockwise screw thread of the second nut with the outer counter-clockwise screw thread of the bolt;
a first supporting member, an end thereof being movably connected to the first nut;
a second supporting member, an end thereof being movably connected to the second nut;
a third supporting member, an end thereof being movably connected to the first nut;
a fourth supporting member, an end thereof being movably connected to the second nut;
a first expanding member, a lateral surface thereof being movably connected to another end of the first supporting member and another end of the second supporting member; and
a second expanding member, a lateral surface thereof being movably connected to another end of the third supporting member and another end of the fourth supporting member,
wherein the bolt is rotated to allow the first nut and the second nut to move in opposite directions along the bolt so that movements of the first supporting member, the second supporting member, the third supporting member and the fourth supporting member drive the first expanding member and the second expanding member to move in opposite directions relative to the lateral surfaces.

2. The vertebral body expanding device according to claim 1, wherein the end of the first supporting member is pivoted to an upper end of the first nut, the end of the second supporting member is pivoted to an upper end of the second nut, the end of the third supporting member is pivoted to a lower end of the first nut, and the end of the fourth supporting member is pivoted to a lower end of the second nut.

3. The vertebral body expanding device according to claim 1, further comprising a transmission member fixed to an end of the bolt, wherein the transmission member comprises an interfering structure for an external tool to engage therewith to exert force on the transmission member so as to rotate the bolt about an axis.

4. The vertebral body expanding device according to claim 3, wherein the interfering structure is disposed on an outer surface of the transmission member.

5. The vertebral body expanding device according to claim 3, wherein the interfering structure is disposed on a lateral wall of a recessed hole formed on the transmission member.

6. The vertebral body expanding device according to claim 3, further comprising a guiding member fixed to the other end of the bolt, wherein the guiding member has a conical shape different from a shape of the other end of the bolt.

7. The vertebral body expanding device according to claim 1, wherein the first expanding member comprises two lateral sides extending toward the second expanding member, and the second expanding member comprises two lateral sides extending toward the first expanding member so as to form an enclosed space.

8. The vertebral body expanding device according to claim 1, wherein the bolt is hollow and further comprises at least one guiding groove.

9. The vertebral body expanding device according to claim 1, wherein the vertebral body expanding device is made of a material selected from a group consisting of polyether ether ketone (PEEK) and titanium alloy.

10. The vertebral body expanding device according to claim 1, wherein the first expanding member comprises at least one rib disposed on an outer surface of the first expanding member, and the second expanding member comprises at least one rib disposed on an outer surface of the second expanding member.

11. A tool set for operating a vertebral body expanding device, the vertebral body expanding device comprising:
a bolt, comprising an outer clockwise screw thread and an outer counter-clockwise screw thread adjacent to the clockwise screw thread;
a first nut, comprising an inner clockwise screw thread and joined to the bolt by engagement of the inner clockwise screw thread of the first nut with the outer clockwise screw thread of the bolt;
a second nut, comprising an inner counter-clockwise screw thread and joined to the bolt by engagement of the inner counter-clockwise screw thread of the second nut with the outer counter-clockwise screw thread of the bolt;
a first supporting member, an end thereof being movably connected to the first nut;
a second supporting member, an end thereof being movably connected to the second nut;
a third supporting member, an end thereof being movably connected to the first nut;
a fourth supporting member, an end thereof being movably connected to the second nut;
a first expanding member, a lateral surface thereof being movably connected to another end of the first supporting member and another end of the second supporting member; and
a second expanding member, a lateral surface thereof being movably connected to another end of the third supporting member and another end of the fourth supporting member,
wherein the bolt is rotated to allow the first nut and the second nut to move in opposite directions along the bolt so that movements of the first supporting member, the second supporting member, the third supporting member and the fourth supporting member drive the first expanding member and the second expanding member to move in opposite directions relative to the lateral surfaces;
and the tool set comprising:
a rotational member, an end thereof being connected to the vertebral body expanding device;
a sleeve surrounding the rotational member, an end thereof being joined to the rotational member and another end thereof being connected to the vertebral body expanding device; and
a grip surrounding the rotational member and the sleeve,
wherein the rotational member is rotated to allow the sleeve to rotate so as to rotate the bolt of the vertebral body expanding device, and the grip does not rotate with the rotational member and the sleeve.

12. The tool set according to claim 11, wherein in the vertebral body expanding device, the end of the first supporting member is pivoted to an upper end of the first nut, the end of the second supporting member is pivoted to an upper end of the second nut, the end of the third supporting member is pivoted to a lower end of the first nut, and the end of the fourth supporting member is pivoted to a lower end of the second nut.

13. The tool set according to claim 11, wherein the vertebral body expanding device further comprises a transmission member fixed to an end of the bolt, wherein the transmission member comprises an interfering structure for an external tool to engage therewith to exert force on the transmission member so as to rotate the bolt about an axis.

14. The tool set according to claim 13, wherein the interfering structure is disposed on an outer surface of the transmission member.

15. The tool set according to claim 13, wherein the interfering structure is disposed on a lateral wall of a recessed hole formed on the transmission member.

16. The tool set according to claim 13, wherein the vertebral body expanding device further comprises a guiding member fixed to the other end of the bolt, wherein the guiding member has a conical shape different from a shape of the other end of the bolt.

17. The tool set according to claim 11, wherein in the vertebral body expanding device, the first expanding member comprises two lateral sides extending toward the second expanding member, and the second expanding member comprises two lateral sides extending toward the first expanding member so as to form an enclosed space.

18. The tool set according to claim 11, wherein the bolt of the vertebral body expanding device is hollow and further comprises at least one guiding groove.

19. The tool set according to claim 11, wherein the vertebral body expanding device is made of a material selected from a group consisting of polyether ether ketone (PEEK) and titanium alloy.

20. The tool set according to claim 11, wherein the first expanding member comprises at least one rib disposed on an outer surface of the first expanding member, and the second expanding member comprises at least one rib disposed on an outer surface of the second expanding member.
